(19) United States
(12) Patent Application Publication
(10) Pub. No.: US 2026/0144771 A1
(43) Pub. Date: May 28, 2026

(54) Use of Triheptanoin Oil for Primary-Specific Pyruvate Dehydrogenase Complex (PDC) Deficiency

(71) Applicant: University of Pittsburgh - Of the Commonwealth System of Higher Education, Pittsburgh, PA (US)

(72) Inventor: Jirair Krikor Bedoyan, Beachwood, OH (US)

(21) Appl. No.: 19/121,545

(22) PCT Filed: Oct. 23, 2023
(86) PCT No.: PCT/US23/35665

§ 371 Date: Apr. 16, 2025

Related U.S. Application Data
(60) Provisional application No. 63/380,832, filed on Oct. 25, 2022.

(51) Int. Cl. A61K 31/225; A61K 31/51; A61P 25/28

(52) U.S. Cl. CPC A61K 31/225; A61K 31/51; A61P 25/28

(57) ABSTRACT

Provided herein is a method of treating a patient having pyruvate dehydrogenase complex deficiency (PDCD), including administering to the patient an amount of triheptanoin effective to treat PDCD in the patient.

### CROSS REFERENCE TO RELATED APPLICATION

The present application claims the benefit of U.S. Provisional Patent Application No. 63/380,832, filed Oct. 25, 2022, the content of which is incorporated herein by reference in its entirety.

### BACKGROUND OF THE INVENTION

### Field of the Invention

Provided herein are methods of treating inherited disorders of energy metabolism, and, in non-limiting embodiments, methods of treating pyruvate dehydrogenase complex deficiency (PDCD) with triheptanoin.

### Description of Related Art

Inherited disorders of energy metabolism include glycogen degradation defects, disorders of gluconeogenesis, disorders of pyruvate metabolism (below), disorders of mitochondrial fatty acid β-oxidation (FAO), tricarboxylic acid (TCA) cycle defects and defects of oxidative phosphorylation, all with either autosomal recessive or X-linked inheritance. Pyruvate dehydrogenase complex deficiency (PDCD) is an inherited disorder of energy metabolism (IDEM) of carbohydrate oxidation mostly affecting the brain and leading to decreased ATP production. At least three groups of PDCDs are recognized with the primary-specific group predominating (˜90% of cases). The main therapy for primary-specific PDCD is life-long ketogenic diet (KD) with oxidation of ketone bodies. However, long-term KD tolerance and maintenance of ketosis with KD, which consists mainly of long- and medium-chain fats, have long been major difficulties for patients with PDCD on KD therapy, thus there is a need in the art for alternative treatments for PDCD.

### SUMMARY OF THE INVENTION

Provided herein is a method of treating a patient having pyruvate dehydrogenase complex deficiency (PDCD), including administering to the patient an amount of triheptanoin effective to treat PDCD in the patient.

Also provided herein is a method of treating a patient having pyruvate dehydrogenase complex deficiency (PDCD), including administering to a patient consuming a ketogenic diet an amount of triheptanoin effective to treat PDCD in the patient, wherein the triheptanoin is titrated across four weeks based on a percentage of the patient's daily intake of calories from fat, wherein, by week, the triheptanoin is administered at a percentage of about 12.5%, about 24.9%, about 37.4%, and about 41.5% of total daily intake of calories from fat.

Also provided herein is a use of triheptanoin for treatment of a patient having a pyruvate dehydrogenase complex deficiency (PDCD).

Non-limiting embodiments of the present disclosure are provided in the following numbered clauses:

Clause 1. A method of treating a patient having pyruvate dehydrogenase complex deficiency (PDCD), comprising administering to the patient an amount of triheptanoin effective to treat PDCD in the patient.

Clause 2. The method of clause 1, wherein treating PDCD in the patient comprises normalizing one or more metabolic parameters in the patient.

Clause 3. The method of clause 1 or clause 2, wherein the metabolic parameter comprises one or more of metabolic acidosis, ketosis, ratio of alanine to leucine, and ratio of proline to leucine.

Clause 4. The method of any of clauses 1-3, wherein the patient is a pediatric patient.

Clause 5. The method of any of clauses 1-4, wherein the patient is on a ketogenic diet.

Clause 6. The method of any of clauses 1-5, wherein the patient does not have a deficit in medium-chain acyl-CoA dehydrogenase.

Clause 7. The method of any of clauses 1-6, wherein the triheptanoin is administered to the patient at a dose of about 1.0 g/kg to about 5 g/kg of body weight per day.

Clause 8. The method of any of clauses 1-7, wherein the triheptanoin is administered to the patient at a dose of about 1.2 g/kg to about 3.9 g/kg of body weight per day.

Clause 9. The method of any of clauses 1-8, wherein the triheptanoin is administered to the patient at a dose of about 4 g/kg of body weight per day.

Clause 10. The method of any of clauses 1-9, wherein administering triheptanoin to the patient comprises titrating the triheptanoin.

Clause 11. The method of any of clauses 1-10, wherein the triheptanoin is titrated over about 28 days.

Clause 12. The method of any of clauses 1-11, wherein the triheptanoin is administered to the patient at a titrated dose of about 1.18 g/kg of body weight per day on days 1-7, at a dose of about 2.37 g/kg of body weight on days 8-14, at a dose of about 3.55 g/kg of body weight on days 15-21, and at a dose of about 3.94 g/kg of body weight on days 22-28.

Clause 13. The method of any of clauses 1-12, wherein the triheptanoin is administered at least three times daily.

Clause 14. The method of any of clauses 1-13, wherein an amount of triheptanoin administered to the patient comprises between about 12.5% and about 41.5% of the patient's daily intake of calories from fat.

Clause 15. The method of any of clauses 1-14, wherein the patient is also administered thiamine at a dose ranging from about 50 mg to about 2000 mg per day.

Clause 16. A method of treating a patient having pyruvate dehydrogenase complex deficiency (PDCD), comprising administering to a patient consuming a ketogenic diet an amount of triheptanoin effective to treat PDCD in the patient, wherein the triheptanoin is titrated across four weeks based on a percentage of the patient's daily intake of calories from fat, wherein, by week, the triheptanoin is administered at a percentage of about 12.5%, about 24.9%, about 37.4%, and about 41.5% of total daily intake of calories from fat.

Clause 17. Use of triheptanoin for treatment of a patient having a pyruvate dehydrogenase complex deficiency (PDCD).

Clause 18. The use of clause 17, wherein treating PDCD in the patient comprises normalizing one or more metabolic parameters in the patient.

Clause 19. The use of clause 17 or clause 18, wherein the metabolic parameter comprises one or more of metabolic acidosis, ketosis, ratio of alanine to leucine, and ratio of proline to leucine.

Clause 20. The use of any of clauses 17-19, wherein the patient is a pediatric patient.

Clause 21. The use of any of clauses 17-20, wherein the patient is on a ketogenic diet.

Clause 22. The use of any of clauses 17-21, wherein the patient does not have a deficit in medium-chain acyl-CoA dehydrogenase.

Clause 23. The use of any of clauses 17-22, wherein the triheptanoin is administered to the patient at a dose of about 1.0 g/kg to about 5 g/kg of body weight.

Clause 24. The use of any of clauses 17-23, wherein the triheptanoin is administered to the patient at a dose of about 1.2 g/kg to about 3.9 g/kg of body weight.

Clause 25. The use of any of clauses 17-24, wherein the triheptanoin is administered to the patient at a dose of about 4 g/kg of body weight per day.

Clause 26. The use of any of clauses 17-25, wherein administering triheptanoin to the patient comprises titrating the triheptanoin.

Clause 27. The use of any of clauses 17-26, wherein the triheptanoin is titrated over about 28 days.

Clause 28. The use of any of clauses 17-27, wherein the triheptanoin is administered to the patient at a titrated dose of about 1.18 g/kg of body weight on days 1-7, at a dose of about 2.37 g/kg of body weight on days 8-14, at a dose of about 3.55 g/kg of body weight on days 15-21, and at a dose of about 3.94 g/kg of body weight on days 22-28.

Clause 29. The use of any of clauses 17-28, wherein the triheptanoin is administered at least three times daily.

Clause 30. The use of any of clauses 17-29, wherein an amount of triheptanoin administered to the patient comprises between about 12.5% and about 41.5% of the patient's daily intake of calories from fat.

Clause 31. The use of any of clauses 17-30, wherein the patient is also administered thiamine at a dose of about 50 mg to about 2000 mg per day.

### DESCRIPTION OF THE INVENTION

The use of numerical values in the various ranges specified in this application, unless expressly indicated otherwise, are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of ranges is intended as a continuous range including every value between the minimum and maximum values. As used herein “a” and “an” refer to one or more.

As used herein, the term “comprising” is open-ended and may be synonymous with “including”, “containing”, or “characterized by”. The term “consisting essentially of” limits the scope of a claim to the specified materials or steps and those that do not materially affect basic and novel characteristic(s). The term “consisting of” excludes any element, step, or ingredient not specified in the claim. As used herein, embodiments “comprising” one or more stated elements or steps also include but are not limited to embodiments “consisting essentially of” and “consisting of” these stated elements or steps.

As used herein, the term “patient” or “subject” refers to members of the animal kingdom including but not limited to human beings and “mammal” refers to all mammals, including, but not limited to human beings.

### Pyruvate Dehydrogenase Complex Disorder

The mitochondrial multienzyme pyruvate dehydrogenase complex (PDC) irreversibly catalyzes the oxidative decarboxylation of pyruvate into acetyl-CoA as the primary substrate for the TCA cycle and oxidative phosphorylation. PDC is comprised of 4 core catalytic subunits (E1α, E1β, E2, and E3 encoded by the PDHA1, PDHB, DLAT and DLD genes, respectively) and a structural protein (E3BP encoded by PDHX). PDC also depends on 4 cofactors (thiamine pyrophosphate, TPP; coenzyme A, CoA; covalently bound lipoate; and flavin adenine dinucleotide, FAD). PDC activity is highly regulated: phosphorylation sites on Ela are regulated by a set of kinases (pyruvate dehydrogenase kinases, PDKs) and phosphatases (pyruvate dehydrogenase phosphatases, PDPs), which interact with lipoyl domains on E2 and E3BP, and are important in inactivation (phosphorylation by kinases) and activation (dephosphorylation by phosphatases) of PDC. The lipoate cofactor is required for catalysis by multiple mitochondrial 2-ketoacid dehydrogenase complexes including PDC, and plays a critical role in stabilizing and regulating PDC function. PDC is also glutathionylated on E2, and this glutathionylation decreases reactive oxygen species (ROS) production when pyruvate is being oxidized, while depletion of glutathione leads to increased ROS production from PDC. Glutathione reductase (GRX2) regulates the reversible glutathionylation, which is important for PDC activity. Sirtuin 4 (SIRT4) regulates PDC function through its lipoamidase activity that cleaves the lipoyl moiety from E2. Defective biosynthesis or mitochondrial transport of co-factors (e.g., thiamine) or substrates (e.g., pyruvate) can also result in functional PDCD. End-product inhibition is yet another mechanism of regulating PDC. The Ki of acetyl-CoA for end-product inhibition of PDC is 5-10 μM, which is much lower than the Ki of propionyl-CoA (3-4 mM), implying that propionyl-CoA has significantly lower affinity for PDC than acetyl-CoA. Furthermore, PDC can translocate from the mitochondria to the nucleus during cell-cycle progression, generating a nuclear pool of acetyl-CoA from pyruvate and increasing the acetylation of core histones important for S phase entry as well as expression of damage response genes among others. Therefore, PDC is a highly regulated mitochondrial matrix multienzyme complex crucial for oxidation of carbohydrates for energy production, but with other “moonlighting” role(s) in cell-cycle progression and potentially cellular differentiation and proliferation.

PDCD is an inherited disorder of energy metabolism (IDEM) because it is a mitochondrial disorder of carbohydrate oxidation that mostly affects the brain and leads to decreased ATP production and energy deficit. PDCD is sub-classified into at least 3 groups, primary-specific, primary-generalized, and secondary PDCD. Mutations in at least 38 genes are associated with PDCD but the majority (80-90%) of genetically resolved PDCD are due to primary-specific PDC genes (PDHA1, PDHB, DLAT, PDHX, and PDP1), with those due to the X-linked PDHA1 (82-88%) predominating. Dysregulation of the ubiquitin-proteasome system can result in E1β instability leading to PDCD but without any molecular defect in PDHB. PDCD is the second most common mitochondrial disease entry within the North American Mitochondrial Disease Consortium (NAMDC) Registry among registry participants. More than 500 cases of PDCDs have been reported, and an estimated at least 1 in 41,000 live births per year in the United States of America (USA) will have primary-specific PDCD (i.e., about 90 newborns annually in USA).

The clinical presentation of PDCD is highly variable and ranges from fatal primary lactic acidosis and congenital brain abnormalities including corpus callosum abnormalities (15-55%), ventriculomegaly (35-85%) and Leigh syndrome (12-25%), to relatively mild ataxia or neuropathy with normal cognitive function and long survival. Epilepsy (16-57%), hypotonia (46-89%), and developmental delay (57-83%) are other common findings in patients with PDCD. The best predictor of survival and cognitive outcome in those affected appears to be age of onset, with neonatal presentations typically associated with early death, and childhood onset ones associated with better survival and with normal or mild to severe cognitive disability. The mean and median ages of diagnosis of childhood onset PDC deficiency are about 45 and 20 months, respectively. Among all the amino acids, only leucine (Leu) and lysine (Lys) are strictly ketogenic amino acids, implying their metabolism would not involve the glycolytic pathway to generate pyruvate but instead would enter the TCA at the acetyl-CoA level bypassing PDC. Because Leu and Lys concentrations are not influenced by the amount of lactate, alanine or proline produced from impaired PDC function, they can be used as normalizing metabolites in quantitative analysis of alanine and proline. A pilot newborn screening (NBS) protocol using a combination of alanine/leucine (Ala/Leu) and proline/leucine (Pro/Leu) ratios for identifying newborns with PDCD using existing NBS analytical approaches for early therapeutic intervention is published (Bedoyan et al., Utility of specific amino acid ratios in screening for pyruvate dehydrogenase complex deficiencies and other mitochondrial disorders associated with congenital lactic acidosis and newborn screening prospects. JIMD Rep. 2020; 56: 70-81). Ala/Leu and Ala/Lys combination ratios are highly sensitive in identifying individuals with PDCD, with 100% sensitive depending on ratio cutoffs, and therefore have utility for future application in PDCD NBS for early identification of at-risk newborns for early intervention.

Life-long use of KD (and avoidance of high carbohydrate diets) is currently the main therapeutic intervention for primary-specific PDCD, with positive outcomes noted in the areas of epilepsy, ataxia, sleep disturbance, speech/language development, social functioning, and frequency of hospitalizations. Although KD therapy has never been evaluated in a clinical trial, its use has the basis of rationale that oxidation of β-hydroxybutyrate and acetoacetate (ketone bodies), which cross the brain barrier and do not depend on PDC for oxidation. Furthermore, oxidation of the ketone bodies provides the brain with an alternative fuel source to generate acetyl-CoA used for maintaining the mitochondrial pools of TCA cycle intermediates and production of reducing potential for subsequent ATP production through the electron transport chain (ETC), thus restoring the energy deficit characteristic of PDCD. Diffusion of ketone bodies across the blood-brain barrier is facilitated by the monocarboxylic transporters.

The efficacy of KD intervention appears to depend at least partially on the disease phenotype and the attainment and maintenance of ketosis. KD can be ineffective in patients with severe brain damage in utero or at birth, and may be harmful (or even lethal) in cases where PDCD is associated with more general impairment of formation of acetyl-CoA (e.g., fatty acid β-oxidation [FAO], branched-chain amino acids [BCAA] metabolism defects or DLD deficiency) or oxidation of acetyl-CoA (e.g., TCA cycle defects such as SUCLA2 deficiency or other mitochondrial dysfunction). Furthermore, KD can be difficult to tolerate long-term, and nausea is reported with KD use, which has been suggested as a manifestation of excess ketosis. The proportions of fat, carbohydrate, and protein in “ketogenic diets” vary considerably in actual practice (e.g., 4:1 or 3:1 fat to carbohydrate and protein calories), with recommendations for minimal (<10%) energy from carbohydrate. “Ketosis” is infrequently monitored by measurement of blood beta-hydroxybutyrate and/or beta-hydroxybutyrate/acetoacetate ratio. Additionally, long-term KD use can be detrimental to lipid metabolism with regard to cholesterol/lipid profiles. Poor cholesterol/lipid profiles in patients on KD presumably can lead to higher risk of cardiovascular disease. A less restrictive partial KD has been used on a select cohort of patients with DLD-E3 deficiency (primary-generalized PDCD) due to a homozygous DLD p.D479V variant, which showed improved survival but no significant improvement in quality of life (QoL).

Supplementation with high dose thiamine (50 to 2000 mg per day) is also used to treat PDCD, with a few case reports of “thiamine responsive” PDCD. Certain pathogenic PDHA1 variants impacting Ela regions that are either not directly participating in TPP binding or have low affinity for TPP, are considered thiamine-responsive. In contrast, the PDHA1 precursor p.R119W variant, where R119 directly binds TPP and is catalytically crucial, is unresponsive to thiamine, at least in vitro in cultured fibroblasts. Thiamine use has not been reported to have adverse side-effects and consequently is commonly administered to patients with PDCD irrespective of responsiveness or efficacy.

Activators of PDC, such as dichloroacetate (DCA), and more recently phenylbutyrate (PB), have been recognized to have potential clinical benefit and have been used or proposed for clinical trials. The mechanism of DCA and PB activation of PDC has been shown to be inhibition of pyruvate dehydrogenase kinases, with different binding sites. Two prior clinical trials of DCA, one controlled, the other open-label, in children with congenital lactic acidosis and adults with various mitochondrial disorders including PDCDs, showed a reduction in cerebrospinal fluid and/or blood lactate, but did not show significant overall clinical benefit. DCA has been associated with toxic neuropathy in subjects with other mitochondrial disorders such as MELAS. A difference between children and adults and an individual association with genetic variations have been shown to affect the rate of catabolism of DCA (and the risk of peripheral neuropathy), which has been proposed as a basis for pharmacokinetic monitoring and management of safe clinical use of DCA. Stimulation of PDC with DCA enhances TCA cycle anabolic bioenergetics in monocytes.

The activity of PDC is regulated through reversible phosphorylation by at least 4 isozymes of PDK with tissue-specific expressions but similar affinities to Ela (Km range 0.1-0.3 mM). PDK1 is the principal isozyme regulating hepatic PDC but it is also readily detectable in all major tissues including the heart; PDK2 is ubiquitously expressed but largely responsible for inactivation of PDC in tissues of muscle origin and brown adipose tissue; and PDK3 regulates PDC in kidney, brain, testis and lung. In contrast, PDK4 level in tissues is low and PDK4 ablation (in mice) has little impact on PDC inactivation. PB inhibits PKD1, PDK2 and PDK3 in vitro and in certain tissues whereas PDK4 is unaffected. In a patient with PDCD harboring the pathogenic p.N135S mutation in PDHA1, PB at 1 mM (and 10 mM) enhanced PDC activity by ˜3.5 (and 4.5)-fold, respectively. Interestingly, PB combined with DCA resulted in significantly greater increase in wild-type or mutant PDC activities compared to each drug alone. In contrast to PB, phenylacetate which is a product of PB metabolism in vivo, does not affect PDC activity in fibroblasts and does not increase PDC activity in brain, muscle and liver of wild-type mice. PB has been used as treatment for patients with maple syrup urine disease (MSUD), which is caused by deficiency of another α-ketoacid dehydrogenase complex—namely the branched-chain α-ketoacid dehydrogenase complex. Collectively, these data suggest that PB alone or in combination with DCA could be effective therapeutic options for certain PDCDs, but this needs further evaluation in clinical trials.

### Triheptanoin

Energy production through carbohydrate oxidation and the electron transport chain (ETC) is dependent upon maintaining mitochondrial pools of TCA cycle intermediates. Even- or odd-chain TCA intermediates may become depleted in subjects with PDCD due to the limited production of acetyl-CoA from carbohydrate oxidation during periods of high-energy demand. Substrates that increase the TCA intermediate pools are termed anaplerotic agents. Anaplerotic therapy has been shown to be a way of restoring depleted TCA cycle intermediates with consequent increase in TCA function and energy metabolism in subjects with long-chain fatty-acid oxidation disorders (LC-FAODs).

Heptanoate (C7:0) diffuses across the mitochondrial membrane much like other medium- and short-chain fats, bypassing the need for the carnitine cycle and the long-chain fatty acid transport, and is oxidized by enzymes of the medium-chain FAO pathway to two acetyl-CoA and one propionyl-CoA. Propionate is anaplerotic and can be converted to succinate by the combined actions of propionyl-CoA carboxylase and methyl-malonyl-CoA mutase. Several studies using triheptanoin as an anaplerotic therapy in metabolic disorders have been published. The biochemical and physical outcomes of subjects with a variety of FAO disorders (FAODs) before and after treatment with triheptanoin show significant improvement in cardiomyopathy and decreased frequency and severity of rhabdomyolysis following supplementation with triheptanoin. A case of pyruvate carboxylase deficiency supplemented also with triheptanoin resulted in rapid and significant improvement of biochemical outcomes. This is consistent with patient outcomes in an open label study on the treatment of patients with pyruvate carboxylase deficiency with triheptanoin. Triheptanoin has also been used for other energy deficit neurometabolic or neurodegenerative conditions such as glucose transporter 1 (Glut1) deficiency and Huntington disease. Furthermore, triheptanoin treatment prevented reduction of PDC and 2-oxoglutarate dehydrogenase activities in a chronic epilepsy mouse model, and partially corrected the reduced incorporation of 13C derived from 13C-glucose in most of the TCA cycle intermediates. Thus, triheptanoin has been successfully used to treat brain and other tissues of energy deficit disorders related to altered anaplerosis, gluconeogenesis and/or glucose metabolism other than PDCD, and thus its usefulness in ameliorating the energy deficit in PDCD is anticipated.

With the foregoing as support, provided herein are methods of treating patients having pyruvate dehydrogenase complex deficiency (PDCD) with a composition including triheptanoin, and uses of compositions including triheptanoin for treatment of the same. In non-limiting embodiments, the triheptanoin is a triheptanoin oil, for example triheptanoin oil sold under the tradename Dojolvi® by Ultragenyx Pharmaceutical, Inc. (Novato, CA).

Triheptanoin is a triglyceride of medium-chain fatty acids, having an odd number chain length of seven carbons. Triheptanoin is a synthetic fatty acid, not produced in nature. Triheptanoin is broken down into glycerol and three heptanoate molecules after ingestion. Heptanoate has the functions described above.

Therapeutic compositions, including those containing triheptanoin, may comprise a pharmaceutically acceptable carrier, or excipient. An excipient is an inactive substance used as a carrier for the active ingredients of a medication. Although “inactive,” excipients may facilitate and aid in increasing the delivery or bioavailability of an active ingredient in a drug product. Non-limiting examples of useful excipients include: antiadherents, binders, rheology modifiers, coatings, disintegrants, emulsifiers, oils, buffers, salts, acids, bases, fillers, diluents, solvents, flavors, colorants, glidants, lubricants, preservatives, antioxidants, sorbents, vitamins, sweeteners, etc., as are available in the pharmaceutical/compounding arts.

Useful dosage forms for triheptanoin compositions include, for example and without limitation: parenteral, intravenous, intramuscular, intraocular, or intraperitoneal solutions, oral tablets or liquids, topical drops, ointments, or creams, and transdermal devices (e.g., patches). The compound may be a sterile solution comprising the active ingredient (drug or compound), and a solvent, such as water, saline, lactated Ringer's solution, or phosphate-buffered saline (PBS). Additional excipients, such as polyethylene glycol, emulsifiers, salts and buffers may be included in the solution. In non-limiting embodiments, triheptanoin as described herein is provided as an oil for an oral route of administration or administration through a feeding tube.

Suitable dosage forms may include single-dose, or multiple-dose vials or other containers, such as medical syringes or droppers. e.g., eye droppers.

Pharmaceutical formulations adapted for administration include aqueous and non-aqueous sterile solutions which may contain, in addition to the active pharmaceutical ingredient or drug, for example and without limitation, anti-oxidants, buffers, bacteriostats, lipids, liposomes, lipid nanoparticles, emulsifiers, suspending agents, and rheology modifiers. The formulations may be presented in unit-dose or multi-dose containers, for example, sealed ampoules and vials, and may be stored in a freeze-dried (lyophilized) condition requiring only the addition of the sterile liquid carrier, for example, water for injections, immediately prior to use. Extemporaneous solutions and suspensions may be prepared from sterile powders, granules and tablets.

Therapeutic/pharmaceutical compositions as described herein may be prepared in accordance with acceptable pharmaceutical procedures, such as described in Remington: The Science and Practice of Pharmacy, 21^{st} edition, ed. Paul Beringer et al., Lippincott, Williams & Wilkins, Baltimore, MD Easton, Pa. (2005) (see, e.g., Chapters 37, 39, 41, 42 and 45 for examples of powder, liquid, parenteral, intravenous and oral solid formulations and methods of making such formulations).

Therapeutic compositions typically must be sterile and stable under the conditions of manufacture and storage. For example, sterile injectable solutions can be prepared by incorporating the active agent in the required amount in an appropriate solvent with one or a combination of ingredients enumerated herein, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, typical methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

Pharmaceutically acceptable salts of the compounds, such as triheptanoin, described herein also may be used in the methods described herein. Pharmaceutically acceptable salt forms of the compounds described herein may be prepared by conventional methods known in the pharmaceutical arts, and include as a class veterinarily-acceptable salts. For example and without limitation, where a compound comprises a carboxylic acid group, a suitable salt thereof may be formed by reacting the compound with an appropriate base to provide the corresponding base addition salt. Non-limiting examples include: alkali metal hydroxides, such as potassium hydroxide, sodium hydroxide and lithium hydroxide; alkaline earth metal hydroxides, such as barium hydroxide and calcium hydroxide; alkali metal alkoxides, such as potassium ethanolate and sodium propanolate; and various organic bases such as piperidine, diethanolamine, and N-methylglutamine.

Non-limiting examples of pharmaceutically-acceptable base salts include: aluminum, ammonium, calcium, copper, ferric, ferrous, lithium, magnesium, manganic, manganous, potassium, sodium, and zinc salts. Salts derived from pharmaceutically acceptable organic non-toxic bases include, without limitation: salts of primary, secondary, and tertiary amines, substituted amines including naturally occurring substituted amines, cyclic amines, and basic ion exchange resins, such as arginine, betaine, caffeine, chloroprocaine, choline, N,N′-dibenzylethylenediamine (benzathine), dicyclohexylamine, diethanolamine, diethylamine, 2-diethylaminoethanol. 2-dimethylaminoethanol, ethanolamine, ethylenediamine, N-ethylmorpholine, N-ethylpiperidine, glucamine, glucosamine, histidine, hydrabamine, iso-propylamine, lidocaine, lysine, meglumine, N-methyl-D-glucamine, morpholine, piperazine, piperidine, polyamine resins, procaine, purines, theobromine, triethanolamine, triethylamine, trimethylamine, tripropylamine, and tris-(hydroxymethyl)-methylamine (tromethamine).

Non-limiting examples of pharmaceutically-acceptable acid salts include: acetate, adipate, alginate, arginate, aspartate, benzoate, besylate (benzenesulfonate), bisulfate, bisulfite, bromide, butyrate, camphorate, camphorsulfonate, caprylate, chloride, chlorobenzoate, citrate, cyclopentanepropionate, digluconate, dihydrogenphosphate, dinitrobenzoate, dodecylsulfate, ethanesulfonate, fumarate, galacterate, galacturonate, glucoheptanoate, gluconate, glutamate, glycerophosphate, hemisuccinate, hemisulfate, heptanoate, hexanoate, hippurate, hydrochloride, hydrobromide, hydroiodide, 2-hydroxyethanesulfonate, iodide, isethionate, iso-butyrate, lactate, lactobionate, malate, maleate, malonate, mandelate, metaphosphate, methanesulfonate, methylbenzoate, monohydrogenphosphate, 2-naphthalenesulfonate, nicotinate, nitrate, oxalate, oleate, pamoate, pectinate, persulfate, phenylacetate, 3-phenylpropionate, phosphate, phosphonate, and phthalate.

Multiple salts forms are also considered to be pharmaceutically-acceptable salts. Common, non-limiting examples of multiple salt forms include: bitartrate, diacetate, difumarate, dimeglumine, diphosphate, disodium, and trihydrochloride.

As such, “pharmaceutically acceptable salt” as used herein is intended to mean an active ingredient (drug) comprising a salt form of any compound as described herein. The salt form may confer improved and/or desirable pharmacokinetic/pharmodynamic properties of the compounds described herein.

A “therapeutically effective amount” refers to an amount of a drug product or active agent effective, at dosages and for periods of time necessary, to achieve the desired therapeutic result. An “amount effective” for treatment of a condition is an amount of an active agent or dosage form, such as a single dose or multiple doses, effective to achieve a determinable end-point. The “amount effective” is preferably safe—at least to the extent the benefits of treatment outweighs the detriments, and/or the detriments are acceptable to one of ordinary skill and/or to an appropriate regulatory agency, such as the U.S. Food and Drug Administration. A therapeutically effective amount of an active agent may vary according to factors such as the disease state, age, sex, and weight of the individual, and the ability of the active agent to elicit a desired response in the individual. A “prophylactically effective amount” refers to an amount effective, at dosages and for periods of time necessary, to achieve a desired prophylactic result. Typically, since a prophylactic dose is used in subjects prior to or at an earlier stage of disease, the prophylactically effective amount may be less than the therapeutically effective amount.

Dosage regimens may be adjusted to provide the optimum desired response (e.g., a therapeutic or prophylactic response). Exemplary dosage regimens are described herein. However, in non-limiting embodiments, a single dose or bolus may be administered, several divided doses may be administered over time, or the composition may be administered continuously or in a pulsed fashion with doses or partial doses being administered at regular intervals, for example, every 10, 15, 20, 30, 45, 60, 90, or 120 minutes, every 2 through 12 hours daily, or every other day, etc., and may be proportionally reduced or increased as indicated by the exigencies of the therapeutic situation. In some instances, it may be especially advantageous to formulate compositions in dosage unit form for case of administration and uniformity of dosage. The specification for the dosage unit forms are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

In non-limiting embodiments, triheptanoin is administered to a patient with PDCD based on body weight of the patient. In non-limiting embodiments, triheptanoin is administered orally and/or through a feeding tube. In non-limiting embodiments, triheptanoin is administered in an amount of between about 0.1 g/kg to about 10 g/kg, about 0.2 g/kg to about 9.9 g/kg, about 0.3 g/kg to about 9.8 g/kg, about 0.4 g/kg to about 9.7 g/kg, about 0.5 to about 9.6 g/kg, about 0.6 g/kg to about 9.5 g/kg, about 0.7 g/kg to about 9.4 g/kg, about 0.8 g/kg to about 9.3 g/kg, about 0.9 g/kg to about 9.2 g/kg, about 1.0 g/kg to about 9.1 g/kg, about 1.1 g/kg to about 9.0 g/kg, about 1.2 g/kg to about 8.9 g/kg, about 1.3 g/kg to about 8.8 g/kg, about 1.4 g/kg to about 8.7 g/kg, about 1.5 g/kg to about 8.6 g/kg, about 1.6 g/kg to about 8.5 g/kg, about 1.7 g/kg to about 8.4 g/kg, about 1.6 g/kg to about 8.3 g/kg, about 1.7 g/kg to about 8.2 g/kg, about 1.8 g/kg to about 8.1 g/kg, about 1.9 g/kg to about 8.0 g/kg, about 2.0 g/kg to about 7.9 g/kg, about 2.1 g/kg to about 7.8 g/kg, about 2.2 g/kg to about 7.7 g/kg, about 2.3 g/kg to about 7.6 g/kg, about 2.4 g/kg to about 7.5 g/kg, about 2.5 g/kg to about 7.4 g/kg, about 2.6 g/kg to about 7.3 g/kg, about 2.7 g/kg to about 7.2 g/kg, about 2.8 g/kg to about 7.1 g/kg, about 2.9 g/kg to about 7.0 g/kg, about 3.0 g/kg to about 6.9 g/kg, about 3.1 g/kg to about 6.8 g/kg, about 3.2 g/kg to about 6.7 g/kg, about 3.3 g/kg to about 6.6 g/kg, about 3.4 g/kg to about 3.5 g/kg, about 3.5 g/kg to about 6.4 g/kg, about 3.6 g/kg to about 6.3 g/kg, about 3.7 g/kg to about 6.2 g/kg, about 3.8 g/kg to about 6.1 g/kg, about 3.9 g/kg to about 6.0 g/kg, about 40. g/kg to about 5.9 g/kg, about 4.1 g/kg to about 5.8 g/kg, about 4.2 g/kg to about 5.7 g/kg, about 4.3 g/kg to about 5.6 g/kg, about 4.4 g/kg to about 5.5 g/kg, about 4.5 g/kg to about 5.4 g/kg, about 4.6 g/kg to about 5.3 g/kg, about 4.7 g/kg to about 5.2 g/kg, about 4.8 g/kg to about 5.1 g/kg, and/or about 4.9 g/kg to about 5.0 g/kg per day, all values and subranges therebetween inclusive, administered in one or more doses per day. In non-limiting embodiments, triheptanoin is administered to the patient in an amount of about 1.2 g/kg of body weight to about 3.9 g/kg of body weight, all values and subranges therebetween inclusive. In non-limiting embodiments, triheptanoin is administered to the patient in an amount of about 4 g/kg per day.

In non-limiting embodiments, triheptanoin is administered to the patient in a titrated dose. By titrated, it is meant that an administered dose of triheptanoin is altered, from an initial dose to a desired end dose, over a course of minutes, hours, days, weeks, months, and/or years. Triheptanoin may contain 8.3 kcals/mL, 8.6 kcals/g and 0.97 g/mL, and titration protocols may be adjusted based on patient age, weight, caloric intake, and/or ketogenic diet. As used herein, a ketogenic diet refers to a diet with a high percentage of fat, moderate protein levels, and low levels of carbohydrates. In non-limiting embodiments, a ketogenic diet refers to a diet in which nutrients are included in the following rages: about 50% to about 70%, optionally about 55% to about 60%, fat, about 25% to about 40%, optionally about 30% to about 35%, protein, and about 1% to about 15%, optionally about 5% to about 10%, carbohydrates, based on a 2000 kcal per day diet, all values and subranges therebetween inclusive. Those of skill in the art are capable of adapting the various percentages disclosed herein based on a different level of caloric intake per day.

In non-limiting embodiments, triheptanoin may be administered in a titration, for any number of days and/or weeks, in any dose described herein. In non-limiting embodiments, triheptanoin is administered in a titrated dose over the course of 1 day, 2 days, 3 days, 4 days, 5 days, 6 days, 7 days, 2 weeks, 3 weeks, 4 weeks, and/or longer, all durations and subranges therebetween inclusive. In non-limiting embodiments, titration, for example for a patient that is 2-years old, with 12.3 kg of body weight and 1250 kcals (102 kcals/kg) of daily caloric intake (DCI) on a most common 4:1 (fat to carbohydrate and protein calories) ketogenic diet with daily 1000 kcals from fat, may be as follows: a DCI of heptanoin of 10, 20, 30 or 35% (goal), which may equate to 125, 250, 375, or 437.5 (goal) kcals daily from triheptanoin, respectively, which may be equal to 15 (1.18), 30 (2.37), 45 (3.55), or 50 (3.94) mL/day of triheptanoin oil (g triheptanoin per kg body weight in parentheses), respectively, which may be divided into at least 3 doses per day. This may translate to 12.5%, 24.9%, 37.4% or 41.5%, of total daily fat calories from triheptanoin, respectively, with sequential titration every 7 days until a final dose goal is reached. In non-limiting embodiments, the goal is 3.94 g/kg of body weight. In non-limiting embodiments, an amount of triheptanoin administered to the patient comprises between about 12.5% and about 41.5% of the patient's daily intake of calories from fat, all values and subranges therebetween inclusive. The daily fat content in a 4:1 ketogenic diet of would be replaced with either triheptanoin in increments of about 125 kcals every 7 days (×3) and then 62.5 kcal once until goal of 35% of DCI from triheptanoin is achieved. However, as noted above, dosages during the titration, and the final dosage, may be selected from any dosage described herein. A non-limiting embodiment of a titration protocol is shown in Table 1, below.

TABLE 1

| | | Daily | | | |
|---|---|---|---|---|---|
| | Daily | Dojolvi* | % DCI | | Dojolvi* |
| | home fat | kcals | from | Duration | g/kg body |
| Timeline | (kcals) | (mL) | Dojolvi* | (days) | weight |
| Before study | 1000 | 0 | 0 | Baseline | None |
| Start of | 875 | 125 (15) | 10% | 7 | 1.18 |
| study | | | | | |
| | 750 | 250 (30) | 20% | 7 | 2.37 |
| | 625 | 375 (45) | 30% | 7 | 3.55 |
| | 562.5 | 438 (50) | 35% | 7 | 3.94 |
| | 562.5 | 438 (50) | 35% | 337 | 3.94 |
| (At 12 | 562.5 | 438 (50) | 35% | Indefinite | 3.94 |
| months) | | | | | |
| Start of | | | | | |
| Maintenance | | | | | |
| (indefinite) | | | | | |

In non-limiting embodiments, the patient's ketogenic diet may be modified based on administration of triheptanoin, in the event that a break in ketosis is observed with the dose escalation during titration (which may be possible based on the glycerol chain of triheptanoin being involved in a conversion to glucose). A non-limiting embodiment of a diet modification is shown in Table 2, below.

TABLE 2

| | | | | | | | Amount of carbs (g) to | | | |
|---|---|---|---|---|---|---|---|---|---|---|
| | | | Fat (g) if X % fat kcals from | | | | decrease from diet if X % | | | |
| | Fat | Total | Dojolvi | | | | fat kcals from Dojolvi ® | | | |
| KD diet | (g) | (g) | 10 | 20 | 30 | 35 | 10 | 20 | 30 | 35 |
| 5:1 | 5.0 | 6.0 | 4.90 | 4.79 | 4.69 | 4.64 | 0.10 | 0.21 | 0.31 | 0.36 |
| | % fat wt in g | 83 | 82 | 80 | 78 | 77 | | | | |
| 4.5:1 | 4.5 | 5.5 | 4.41 | 4.31 | 4.22 | 4.17 | 0.09 | 0.19 | 0.28 | 0.33 |
| | % fat wt in g | 82 | 80 | 78 | 77 | 76 | | | | |
| 4:1 | 4.0 | 5.0 | 3.92 | 3.83 | 3.75 | 3.71 | 0.08 | 0.17 | 0.25 | 0.29 |
| | % fat wt in g | 80 | 78 | 77 | 75 | 74 | | | | |
| 3.5:1 | 3.5 | 4.5 | 3.43 | 3.35 | 3.28 | 3.25 | 0.07 | 0.15 | 0.22 | 0.25 |
| | % fat wt in g | 78 | 76 | 75 | 73 | 72 | | | | |
| 3:1 | 3.0 | 4.0 | 2.94 | 2.88 | 2.81 | 2.78 | 0.08 | 0.12 | 0.19 | 0.22 |
| | % fat wt in g | 75 | 73 | 72 | 70 | 70 | | | | |
| 2.5:1 | 2.5 | 3.5 | 2.45 | 2.40 | 2.34 | 2.32 | 0.05 | 0.10 | 0.16 | 0.18 |
| | % fat wt in g | 71 | 70 | 68 | 67 | 66 | | | | |

For example, if a break with ketosis is noted in a subject originally on a 4:1 KD after 10% fat kcals replacement with triheptanoin, a patient could switch to a 4.5:1 KD with 10% fat kcals from triheptanoin, with maintenance of equivalent % fat wt in grams or could reduce carbohydrate intake by 0.08 g and remain on a 4:1 KD. That is, in the event that there is a break in ketosis in a patient receiving triheptanoin, treatment could be adjusted by increasing the strength of the ketogenic diet or reducing carbohydrate intake, thus allowing the patient to continue to receive triheptanoin.

In non-limiting embodiments, the patient to whom triheptanoin is administered is a pediatric patient. In non-limiting embodiments, the patient is between the ages of 1 month and 18 years old, all ages and subranges therebetween inclusive. In non-limiting embodiments, the patient is determined to have PDCD based on molecular genetic confirmation of PDHA1, PDHB, DLAT, PDHX, and/or PDP1 mutation. In non-limiting embodiments, the patient does not have a deficiency in medium-chain acyl-CoA dehydrogenase (MCAD), from mutations in the ACADM gene besides the common c.985A>G mutation as one of allele mutations. In non-limiting embodiments, the patient also has a deficiency in MCAD.

Administration of triheptanoin is expected to have beneficial effects in a number of parameters, including clinical and laboratory parameters. In non-limiting embodiments, administration of triheptanoin to patients with PDCD results in:
same or more effective control of metabolic acidosis including blood lactate and pyruvate levels;
same or more effective maintenance of ketosis including blood β-hydroxybutyrate (BOHB) level, for example, a level of between about 1 mM and about 7 mM, between about 1 mM and about 4.5 mM, between about 1 mM and about 2 mM, between about 3.5 mM and about 4.5 mM, all values and subranges therebetween inclusive;
same or improved blood Ala/Leu, Ala/Lys, Pro/Leu, and/or (Ala+Pro)/(Leu+Lys) ratios (biomarkers of degree of metabolic control in PDCD, for example, an Ala/Leu of <5.0 and/or <4.0, an Ala/Lys of <3.0, a Pro/Leu of <3.0, and/or an (Ala+Pro)/(Leu+Lys) of <2.5, all values and subranges therebetween inclusive);
same or more efficacious clinical control including:
same or more efficacious seizure control with reduction or alteration of home antiepileptics use;
same or fewer episodes of metabolic decompensation;
same or decreased frequency and duration of hospitalizations;
improvements in quality of life as measured by:
PedsQL™ (Family Impact Module; total, parent-reported health-related QoL (HRQoL), and family functioning scores completed by parent/caregiver);
MetabQoL 1.0 (total, physical, mental, and social scores completed by a psychometrician for individuals up to 18 years of age); and/or
better long-term maintenance and tolerance of diet per parental reporting.

In non-limiting embodiments, additional parameters that may be measured, and which may be improved based on administration of triheptanoin, include bicarbonate level, ketosis, propionate, lactate and pyruvate levels, Ala/Leu, Ala/Lys, Pro/Leu and Pro/Lys ratios, acetyl- and propionyl-carnitine levels, basic electrolytes and blood count perturbations, hepatic and renal function parameters, and/or progression or improvement of neurometabolic disorders.

In non-limiting embodiments, a patient to whom triheptanoin is administered, in addition and/or as an alternative to a ketogenic diet, may also be treated with thiamine. In non-limiting embodiments, the patient is administered between about 50 mg to about 2000 mg per day of thiamine, all values and subranges therebetween inclusive.

### EXAMPLE

The current study is a single-center, investigator-initiated, exploratory proof-of-concept study to evaluate efficacy of triheptanoin (Dojolvi®) for use in patients with primary-specific PDCD on a ketogenic diet (KD). The primary working hypothesis of this study is that triheptanoin will be efficacious for patients with PDCD to restore TCA cycle deficit and in substitution of the usual fats including medium- or long-chain triglycerides (MCT/LCT) as part of KD for PDCD patients on such therapy. The expected result would be to restore the energy deficit inherent with this disorder, for better outcome, quality of life and survival.

### Outcome Measures and Testing

The efficacy of Dojolvi® use for PDCD patients on KD will be evaluated. This efficacy evaluation is for exploratory purposes only and will be pursued as follows:
Primary endpoints: Safety and efficacy of Dojolvi® will be evaluated in patients with PDCD on KD, specifically, whether Dojolvi®:
is safely tolerated with the exception of its commonly reported side-effect of some GI distress;
provides same or improved biomarkers and measures of disease including:
same or more effective control of metabolic acidosis including blood lactate and pyruvate levels;
same or more effective maintenance of ketosis including blood β-hydroxybutyrate level;
same or improved blood Ala/Leu, Ala/Lys and Pro/Leu ratios (biomarkers of degree of metabolic control in PDCD);
leads to same or more efficacious clinical control including:
same or more efficacious seizure control with reduction or alteration of home antiepileptics use;
same or fewer episodes of metabolic decompensation;
same or decreased frequency and duration of hospitalizations;

Secondary endpoints: Efficacy of Dojolvi® for patients with PDCD on KD will be further evaluated by whether its use also improves quality of life as measured by:
PedsQL™ (Family Impact Module; total, parent-reported health-related QoL (HRQoL), and family functioning scores completed by parent/caregiver); and
MetabQoL 1.0 (total, physical, mental, and social scores completed by a psychometrician for individuals up to 18 years of age)
leads to better long-term maintenance and tolerance of diet per parental reporting.

Safety and efficacy will be evaluated through clinical and laboratory parameters (Table 3, below).

TABLE 3

| | Screening | Intake | | | | | | | |
|---|---|---|---|---|---|---|---|---|---|
| | Visit | & | Week 1, | | Months 2 | | | | Maint. |
| | (Remote) | Dosing | 2, and 3 [symbol] | Month 1 | and 3 [symbol] | Month 6 | Month 9 [symbol] | Month 12 | (6 months) |
| Procedure | (35 Days) | (Day 1) | (±2 Days) | (±7 Days) | (±7 Days) | (±7 Days) | (±7 Days) | (±7 Days) | (±7 Days) |
| Informed Consent | X [symbol] | | | | | | | | |
| Complete history (including | | X | | | | | | | |
| demographics) | | | | | | | | | |
| Interim history | | | | X | | X | | X | X |
| Nutritional history/diet diary | X [symbol] | | | X | | X | | X | X |
| Parent Nutritional Survey | | X | | X | | X | | X | X |
| Complete physical | | X | | | | X | | X | X |
| Weight² | | X | | X [symbol] | | X | | X | X |
| Height | | X | | X [symbol] | | X | | X | X |
| Vital Signs | | X | | X [symbol] | | X | | X | X |
| CMP | | X | X | X [symbol] | X | X | X | X | X |
| Pregnancy test [symbol] | | X | | X [symbol] | | X | | X | X |
| Blood beta-hydroxybutyrate | | X | X | X [symbol] | X | X | X | X | X |
| Lactate and pyruvate | | X | X | X [symbol] | X | X | X | X | X |
| TSH and | | X | | X [symbol] | | X | | X | X |
| free-T4 | | | | | | | | | |
| Thiamine and TFP | | X | | X [symbol] | | X | | X | X |
| Plasma Acylamino profile | | X | X | X [symbol] | X | X | X | X | X |
| [symbol] battery | | X | X | X [symbol] | X | X | X | X | X |
| Lipid profile | | X | | X [symbol] | | X | | X | X |
| Essential fatty acid profile | | X | | X [symbol] | | X | | X | X |
| [symbol] Amino Acids | | X | X | X [symbol] | X | X | X | X | X |
| [symbol] | | X | | X [symbol] | | X | | X | X |
| Iron. TiBG and % saturation | | X | | X [symbol] | | X | | X | X |
| CBC with differential | | X | | X [symbol] | | X | | X | X |
| Urine organic acids | | X | X | X [symbol] | X | X | X | X | X |
| Urine analysis | | X | X | X [symbol] | X | X | X | X | X |
| Quality of life (QoL) survey | | X | | X [symbol] | | X | | X | X |
| PedsQL ™ and [symbol] | | | | | | | | | |
| Adverse Events (AE) [symbol] | | Weekly and then monthly call with Study Coordinator/Dietician | | | | | | | |
| Concomitant Medications [symbol] | | Weekly and then monthly call with Study Coordinator/Dietitian | | | | | | | |
| Questionnaire for movement | | Weekly and then monthly call with Study Coordinator/Dietitian | | | | | | | |
| changes [symbol] | | | | | | | | | |
| Study Drug Dispensing and | | X | | X | | X | | X | X |
| Accountability | | | | | | | | | |
| Weekly Call with Study | | Weekly call with Study Coordinator/Dietician during dose titration | | | | | | | |
| Coordinator/Dietician [symbol] | | | | | | | | | |
| Monthly Call with Study | | Ongoing Monthly call with Study Coordinator/Dietician | | | | | | | |
| Coordinator [symbol] | | | | | | | | | |

[symbol]

indicates data missing or illegible when filed

Patients will be monitored for known associated complications of PDCD on KD including KD tolerance and maintenance issues, bicarbonate level, ketosis, propionate, lactate and pyruvate levels, Ala/Leu, Ala/Lys, Pro/Leu and Pro/Lys ratios, acetyl- and propionyl-carnitine levels, basic electrolytes and blood count perturbations, hepatic and renal function parameters, and progression or improvement of the neurometabolic disorder while on therapy. Data obtained through this protocol will allow determination of efficiency and the relative requirement of triheptanoin (Dojolvi®) for modifying KD for infants and older children, and identification of toxicity if any to liver and kidney when used in conjunction with KD.

### Patient Population and Inclusion Criteria

Pediatric patients aged birth to <18 years of age with primary-specific PDCD will be enrolled in this study. Patients with PDCD would need to have a metabolic physician following their clinical care needs prior to their enrollment in the study. The diagnosis and molecular confirmation for each PDCD patient will be clearly documented prior to inclusion in the study. Eligible patients will be limited to those with primary-specific PDCD due to either PDHA1, PDHB, DLAT, PDHX, or PDP1, with PDHA1 cases expected to predominate. No other patients will qualify for entry into the study protocol. Women who are pregnant will not be eligible for the study. Patients or parent/guardian will be required to consent for this study and to having all blood and urine results monitored during their routine care to be reviewed and tabulated for the protocol.
Age 1 year to <18 years of age
Subjects with PDCD would need to have a metabolic physician following their clinical care needs prior to their enrollment in the study
Diagnosis of PDCD by molecular genetic confirmation of PDHA1, PDHB, DLAT, PDHX, or PDP1 mutation
Not pregnant or lactating
Parental permission and assent of minor and willingness to comply with study procedures
Not participating in any interventional treatment clinical trials
Not a recipient of gene therapy, organ transplant, or bone-marrow transplantation
If currently on any investigational drugs or therapies, must complete a 30-day washout period prior to Intake & Dosing (Day 1)
Negative pregnancy test for all female patients of childbearing age. Individuals of childbearing potential must agree to use a highly effective method of contraception, and males must agree not to father a child or donate sperm. True abstinence for the duration of the study will also be accepted

Subjects are following some form or type of ketogenic diet at the time of the screening visit.

### Exclusion Criteria

Medium-chain acyl-CoA dehydrogenase (MCAD) is required for triheptanoin metabolism. Consequently, individuals with a known biochemically or molecularly confirmed deficiency of MCAD will be excluded from this study. The use of alcohol, other recreational drugs of abuse, or marijuana by prescription may interfere with the evaluation of benefits from this treatment protocol and their detection will result in exclusion of the patient from the protocol. Pregnant women will not be enrolled in the protocol.
Diagnosis of medium-chain acyl-CoA dehydrogenase (MCAD)
Use of alcohol or drugs of abuse
Evidence of liver disease as defined by elevations of AST or ALT>1.5×ULN in the past 6 months
Pregnant, breastfeeding, or lactating females
On any investigational product research study (and not completed the required 30-day washout period prior to Intake & Dosing) or recipient of gene therapy or organ or bone-marrow transplantation

### Study Type

This will be an exploratory, proof-of concept study protocol using triheptanoin (Dojolvi®). Pediatric patients (aged birth to <18 years of age) with primary-specific PDCD due to pathogenic mutations in either PDHA1, PDHB, DLAT, PDHX, or PDP1 will be enrolled on a rolling basis.

### Stopping Criteria

Patients who experience NCI CTCAE Grade 3 (severe) or higher adverse events during the study will be withdrawn from the study rather than down titrating the Dojolvi® dose even if they had tolerated well the previous Dojolvi® dose, and the study will be stopped if 2 patients developed the same Grade 3 (severe) Adverse Event, or for any patient that develops a Grade 4 (life-threatening) Adverse Event based on NCI CTCAE. Any patient reporting a pregnancy will be immediately removed from the study.

### Subject Withdrawal Criteria

Patients may be withdrawn for any of the following reasons:
Voluntary withdrawal
Noncompliance as determined by the investigator
At the discretion of the investigator if it is in the best interest of the patient
Meeting any of the patient stopping rules in Section 3.5
Lost to follow up

### Data Safety and Monitoring Plan

There will be an evaluation of the progress of the research study, including assessments of data quality, timelines, participant recruitment, accrual, and retention. The Investigator will also review the outcome and adverse event data to determine whether there is any change to the anticipated benefit-to-risk ratio of study participation and whether the study should continue as originally designed or be re-evaluated and changed. A summary report of data and safety monitoring meetings will be provided to the IRB at the time of the continuing review.

### Parameters to be Monitored

The following progress will be monitored throughout the course of the research to ensure the safety of patients as well as the integrity and confidentiality of their data:

An evaluation of the progress of the research study, including patient recruitment and retention, and an assessment of the timeliness and quality of the data.

A review of collected data (including adverse events, unanticipated problems requiring reporting and those captured on the non-compliance log, and patient withdrawals) to determine whether there is a change to the anticipated benefit-to-risk assessment of study participation and whether the study should continue as originally designed, should be changed, or should be terminated.

An assessment of external factors or relevant information (e.g. pertinent scientific literature reports or therapeutic development, results of related studies) that may have an impact on the safety and study participants or the ethics of the research study.

A review of study procedures designed to protect the privacy of the research patients and the confidentiality of their research data.

### Frequency of Monitoring

The Investigator will review patient safety data as it is reported and documented. The Investigator, sub-investigators, and the research staff will meet on a monthly basis to review patient recruitment, data, source documentation and identification of adverse events, complaints and confidentiality of patients.

### Clinical Monitoring

In accordance with 21 C.F.R. § 312.50, clinical site monitoring will be conducted to ensure that the rights and well-being of trial participants are protected, that the reported trial data are accurate, complete, and that the conduct of the trial is in compliance with currently approved protocol/amendment(s).

Independent monitoring of the clinical study for protocol and GCP compliance will be conducted periodically (i.e., at a minimum of annually) by qualified staff of the Education and Compliance Support for Human Subject Research (ECS-HSR) Division, University of Pittsburgh.

The Sponsor-Investigator and the University of Pittsburgh and UPMC will permit direct access of the study monitors and appropriate regulatory authorities to the study data and to the corresponding source data and documents to verify the accuracy of this data.

The coordinator(s) will review data at least monthly with sponsor-investigator to discuss accrual, unanticipated problems, and adverse events. Serious adverse events will be addressed immediately. The sponsor-investigator is always available to study staff to discuss any adverse events or other concerns as needed.

### Data and Safety Monitor

A data safety monitor from the study will be appointed prior to beginning the protocol. The data safety monitor will be a senior faculty member in the Department of Pediatrics at the University of Pittsburgh School of Medicine who is familiar with PDCD but is not a member of the Division of Genetic and Genomic Medicine. All adverse events will be recorded in the patient's study record and reported within 24 hours to the data safety monitor. Serious adverse events (SAEs), defined as any untoward medical occurrence that is life-threatening, requires inpatient hospitalization or prolongation of existing hospitalization, results in persistent or significant disability/incapacity, results in death, or is a congenital anomaly/birth defect will be reported immediately regardless of relationship to treatment. Monitoring for specific potential adverse events with use of triheptanoin will include measurement of propionate, acetyl- and propionyl-carnitine levels as noted above (Section 3.1). Study Treatment and Laboratory Testing Plan.

All patients will be evaluated by the study PI or sub-Investigator (Sub-I) on admission to this program. Standard of care laboratory testing listed in Table 3 above will be performed.

Patients will be contacted by the study coordinator to schedule a remote screening visit via Microsoft Teams. During this call, the PI and study coordinator will review the consent and answer any questions about the study. The patient will sign the consent electronically via 21 C.F.R. § 11 compliant DocuSign and follow the Division of Genetic and Genomic Medicine's remote consent standard operating procedure. The patient will complete a 3-day diet diary and provide it to the study metabolic dietitian at least 7 days prior to Intake (Day 1) to allow the dietitian adequate time for review and dose calculation. Within 14-21 days of signing the consent, the patient will come to the Division of Genetic and Genomic Medicine at the UPMC Children's Hospital of Pittsburgh. At this visit, the assessments listed in the schedule of events table (Table 1) will be completed.

Following analysis of their home ketogenic diet, provided during the screening visit, from actual intake and review of recent clinic diet records by a dietitian, and a negative pregnancy test, if applicable, patients will receive a modified ketogenic diet containing measured amounts of Dojolvi® with titration over 28 days to goal (Table 1, above). Dietitian will substitute fat calories from food intake or within the ketogenic diet with equivalent fat calories from Dojolvi®. The investigation will target 1.2-3.9 g of Dojolvi® per kg body weight in KD with max goal of about 4 g/kg per day. Dojolvi® is 8.3 kcals/mL, 8.6 kcals/g and 0.97 g/mL. An example of Dojolvi® protocol titration for a 2-year-old boy with 12.3 kg of body weight (˜50% ile) and 1250 kcals (102 kcals/kg) of daily caloric intake (DCI) on most common 4:1 (fat to carbohydrate and protein calories) ketogenic diet with daily 1000 kcals from fat, would be as follows: A Dojolvi® DCI of 10, 20, 30 or 35% (goal) implies 125, 250, 375, or 437.5 (goal) kcals daily from triheptanoin, respectively, which is equal to 15 (1.18), 30 (2.37), 45 (3.55), or 50 (3.94) mL/day of Dojovli® (g Dojolvi® per kg body weight), respectively, to be divided into at least 3 times per day. This translates to 12.5%, 24.9%, 37.4% or 41.5% total daily fat calories from Dojolvi®, respectively, with sequential titration every 7 days until goal (3.94 g/kg) is reached. The daily fat content in the 4:1 ketogenic diet of this individual will be replaced with either Dojolvi® by increments of about 125 kcals every 7 days ×3 and then 62.5 kcal once until goal of 35% of DCI from Dojolvi® is achieved (Table 1, above, represents a Dojolvi® titration example for a specific patient on a 4:1 ketogenic diet). This will be given via G-tube infusion (mixed in with formula) or orally divided into 3-4 doses per Dojolvi® Patient Information Insert recommendations. Dojolvi® (triheptanoin) should be administered at least 4 times per day orally diluted with foods, liquids, or formula via a silicone or polyurethane feeding tube. The dose will be adjusted based on safety laboratory monitoring at Month 1 as outlined in Table 1 and for adverse symptoms. The most commonly reported side effect of the medication (≥10% of cases) has been GI distress. In the event of GI distress (cramping, diarrhea, vomiting, or nausea), the dose of Dojolvi® will be decreased in increments of 5% until symptom(s) resolve. However, patients with Grade 3 GI CTCAE will be removed and the dose won't be decreased to a tolerated dose.

The development of propionic acidemia will be monitored through measurement of blood acylcarnitine profile and urine organic acids. If significant levels accumulate (greater than 1.5× the upper limit of normal (ULN) for the testing laboratory), Dojolvi® dosing will be decreased incrementally by 5% until the level returns to an acceptable range. The remainder of their diet will be modified to maintain appropriate caloric intake and balance of fat to carbohydrate and protein kcals ratio per their usual home ketogenic diet. No more than 35% of total DCI will come from triheptanoin as currently recommended in the Dojolvi® PI. If a patient is unable to achieve the target daily dosage of up to 35% DCI after dosage titration, the patient will be maintained at the maximum tolerated dosage.

Patients will return to the Division of Genetic and Genomic Medicine at the UPMC Children's Hospital of Pittsburgh to be monitored for development of any untoward effects at Months 6 and 12. Laboratory evaluations will take place with samples for acylcarnitine and organic acid analysis as well as other labs being obtained on each occasion (Table 3, above). Patients will monitor their weight at home on a monthly basis. Parents will be instructed to collect a weekly body weight and contact study staff anytime there is a ±20% change. The study team will assess if a ketogenic diet modification is required. Patient/family will be contacted via teleconference with site staff weekly for the first four weeks after starting Dojolvi®, and then monthly thereafter. Any changes in movement events (including seizure or seizure-like activities with any abnormal posturing, myoclonus, spasticity, or eye or mouth movements) will be noted by research coordination/dietitian. Any such changes from baseline will prompt a call to the individual's neurologist for evaluation and indicated as an adverse event (AE). If such movement changes trigger a hospital admission, then this will be noted as a severe adverse event (SAE). Quality-of-life (QoL) surveys (parent- and provider-based ones) will be administered at Intake (Day 1), at Months 1, 6, and 12, and every 6 months thereafter. Following completion of the 12-month of treatment, patients will be placed on a continuing schedule for maintenance of triheptanoin (Dojolvi®) therapy if the results showed it was beneficial. If patients decide to continue triheptanoin, they will need to complete clinical visits every 6 months. They will come to the site to complete assessments per schedule of events (Table 2, above). In addition to the timepoints noted above, the patient will be asked to have laboratory assessments collected at weeks 1, 2, and 3 and months 2, 3, and 9 after starting Dojolvi®. These can be done at the UPMC Children's Hospital of Pittsburgh or at a local laboratory. All laboratory testing will be completed as standard of care (SOC).

Triheptanoin oil (Dojolvi®) will be supplied by Ultragenyx in 500 mL amber glass bottles at no cost for patients in this program. The Dojolvi® will be provided to the research pharmacy which will package as appropriate for each patient's dose. Patients will be asked to return empty bottles or unused product to assess for compliance.

The research coordinator will administer the provider Quality of Life (QoL) surveys. The metabolic dietitian will be adding the appropriate amount of Dojolvi® oil to ketogenic diet per guidance provided by the PI. Ultragenyx will provide 500 ml amber glass bottles of Dojolvi®.

### Treatment Modification for Break in Ketosis (BIK)

Clinicians of patients with PDCD on ketogenic diet have had good success with supplementing some PDCD patients with MCT (personal experience and experience of other clinicians). MCT oil contains a glycerol chain like Dojolvi® and presumably this glycerol could in principle be involved in conversion to glucose, in which case one might note a break in ketosis (BIK) in patients with PDCD on ketogenic diet with escalation of Dojolvi® content in ketogenic diet. However, this is conjectural and remains to be determined and reported. Furthermore, the accumulating oxaloacetate (a substrate for phosphoenolpyruvate carboxykinase to generate phosphoenolpyruvate) from the anaplerotic effect of Dojolvi® via propionyl-CoA generation may be an alternate reason for BIK. Ketones and lactate will be carefully monitored in study patients for any signs of BIK. A different strength of ketogenic diet will be adopted in a patient on ketogenic diet if ketosis is broken with Dojolvi® use. Table 2 above shows ketogenic diet modification (or alternatively, an equivalent reduction in daily carbohydrate intake), that could be adopted with Dojolvi® escalation if BIK is noted. If BIK is still observed even with diet modification (for example, as in Table 2, above), then the accumulating oxaloacetate from the anaplerotic effect of Dojolvi® would most likely be the reason for BIK, warranting further investigations. This would be a valuable observation for investigators and clinicians using MCT oil to supplement ketogenic diet or using Dojolvi® for other disorders of energy deficit such as Glut1, where patients also are commonly placed on ketogenic diet.

The present invention has been described with reference to certain exemplary embodiments, dispersible compositions and uses thereof. However, it will be recognized by those of ordinary skill in the art that various substitutions, modifications or combinations of any of the exemplary embodiments may be made without departing from the spirit and scope of the invention. Thus, the invention is not limited by the description of the exemplary embodiments, but rather by the appended claims as originally filed.

1. A method of treating a patient having pyruvate dehydrogenase complex deficiency (PDCD), comprising administering to the patient an amount of triheptanoin effective to treat PDCD in the patient.

2. The method of claim 1, wherein treating PDCD in the patient comprises normalizing one or more metabolic parameters in the patient.

3. The method of claim 2, wherein the metabolic parameter comprises one or more of metabolic acidosis, ketosis, ratio of alanine to leucine, and ratio of proline to leucine.

4. The method of claim 1, wherein the patient is a pediatric patient.

5. The method of claim 1, wherein the patient is on a ketogenic diet.

6. The method of claim 1, wherein the patient does not have a deficit in medium-chain acyl-CoA dehydrogenase.

7. The method of claim 1, wherein the triheptanoin is administered to the patient at a dose of about 1.0 g/kg to about 5 g/kg of body weight per day.

8. The method of claim 1, wherein the triheptanoin is administered to the patient at a dose of about 1.2 g/kg to about 3.9 g/kg of body weight per day.

9. The method of claim 1, wherein the triheptanoin is administered to the patient at a dose of about 4 g/kg of body weight per day.

10. The method of claim 1, wherein administering triheptanoin to the patient comprises titrating the triheptanoin.

11. The method of claim 10, wherein the triheptanoin is titrated over about 28 days.

12. The method of claim 10, wherein the triheptanoin is administered to the patient at a titrated dose of about 1.18 g/kg of body weight per day on days 1-7, at a dose of about 2.37 g/kg of body weight on days 8-14, at a dose of about 3.55 g/kg of body weight on days 15-21, and at a dose of about 3.94 g/kg of body weight on days 22-28.

13. The method of claim 12, wherein the triheptanoin is administered at least three times daily

14. The method of claim 1, wherein an amount of triheptanoin administered to the patient comprises between about 12.5% and about 41.5% of the patient's daily intake of calories from fat

15. The method of claim 1, wherein the patient is also administered thiamine at a dose ranging from about 50 mg to about 2000 mg per day.

16. A method of treating a patient having pyruvate dehydrogenase complex deficiency (PDCD), comprising administering to a patient consuming a ketogenic diet an amount of triheptanoin effective to treat PDCD in the patient, wherein the triheptanoin is titrated across four weeks based on a percentage of the patient's daily intake of calories from fat, wherein, by week, the triheptanoin is administered at a percentage of about 12.5%, about 24.9%, about 37.4%, and about 41.5% of total daily intake of calories from fat.

17-31. (canceled)
